(19) United States
(12) Patent Application Publication
(10) Pub. No.: US 2010/0260465 A1
(43) Pub. Date: Oct. 14, 2010

(54) Protein engineering strategies to optimize activity of surface attached proteins

(75) Inventors: David Hanzel, Palo Alto, CA (US); Jonas Korlach, Menlo Park, CA (US); Paul Peluso, Hayward, CA (US); Geoff Otto, Santa Clara, CA (US); Thang Pham, Mountain View, CA (US); David Rank, Palo Alto, CA (US); Stephen Turner, Menlo Park, CA (US)

Correspondence Address: QUINE INTELLECTUAL PROPERTY LAW GROUP, P.C., P O BOX 458, ALAMEDA, CA, 94501, US

(73) Assignee: Pacific Biosciences of California, Inc., Menlo Park, CA (US)

(21) Appl. No.: 11/645,135

(22) Filed: Dec. 21, 2006

Related U.S. Application Data
(60) Provisional application No. 60/753,446, filed on Dec. 22, 2005.

(51) Int. Cl. G02B 6/00; C12N 9/00; C12N 9/10; C12N 9/48; C12N 9/16; C12N 9/02; C12N 9/84; C12N 9/38; C07K 14/00; B32B 1/00; B32B 17/06; B32B 18/00; B32B 9/00

(52) U.S. Cl. 385/141; 435/183; 435/193; 435/212; 435/196; 435/189; 435/230; 435/207; 530/350; 428/403; 428/404; 428/406; 428/426; 428/447; 428/478.2

(57) ABSTRACT

Isolated and/or recombinant enzymes that include surface binding domains, surfaces with active enzymes bound to them and methods of coupling enzymes to surfaces are provided. Enzymes can include large and/or multiple surface coupling domains for surface coupling.

### CROSS-REFERENCE TO RELATED APPLICATIONS

This application is a non-provisional utility patent application claiming priority to and benefit of the following prior provisional patent application: U.S. Ser. No. 60/753,446, filed Dec. 22, 2005, entitled “PROTEIN ENGINEERING STRATEGIES TO OPTIMIZE ACTIVITY OF SURFACE ATTACHED PROTEINS” by David Hanzel et al., which is incorporated herein by reference in its entirety for all purposes.

### FIELD OF THE INVENTION

The present invention relates to enzymes comprising surface binding domains and surfaces with active enzymes bound to them. Methods of coupling enzymes to surfaces are also described.

### BACKGROUND OF THE INVENTION

Assays that detect activity of surface-bound polypeptides are common. For example, arrays of polypeptides are commonly assayed for binding to an analyte of interest. Such arrays of polypeptides are often made synthetically on the surface itself, e.g., through combinatorial solid-phase synthesis methods.

Polypeptides can also be made recombinantly and subsequently coupled to a surface for further analysis. Commonly, this is done with a covalent interaction between the protein and a surface, e.g., as in typical plasmon resonance applications. Proteins can also be coupled through various affinity tags, e.g., antibodies such as anti-HA can be bound to a surface and complexed with an HA-tagged fusion protein. Similarly, a His-tagged protein can be captured on a surface that comprises a nickel-NTA moiety (the His residues coordinate with the nickel on the surface). For example, Nieba et al. (1997) “BIACORE analysis of histidine-tagged proteins using a chelating NTA sensor chip” Analytical Biochemistry 252: 217-228, describe BIAcore® analysis of the interaction between various His-tagged protein constructs and a nickel-NTA sensor chip.

Thus, several attachment methods for attaching proteins to surfaces are known. However, strategies for attaching proteins to surfaces often suffer from a variety of problems, including non-specific protein binding to the surface (e.g., due to charge interactions), denaturation of the proteins on the surfaces, due to surface effects, and inaccessibility of protein active sites on the surfaces, due to incorrect orientation of the protein with respect to the surface and/or denaturation of the protein on surfaces.

A variety of technologies have been developed to address some of these issues. For example, proteins have been attached to glass surfaces by copolymerization with a polyacrylamide hydrogel. See, e.g., Brueggemeier et al. (2005) “Protein-Acrylamide Copolymer Hydrogels for Array-Based Detection of Tyrosine Kinase Activity from Cell Lysates” Biomacromolecules 6(5): 2765-2775. In this approach, Glutathione S-transferase-Crkl (GST-Crkl) fusion proteins were covalently immobilized on polyacrylamide gel pads via copolymerization of acrylic monomer and acrylic-functionalized GST-Crkl protein constructs. The resulting hydrogels resist nonspecific protein adsorption. However, this technology results in the protein being attached in several different orientations to the surface, with the protein's active site being inconsistently presented to a solution phase. This makes analysis of single bound proteins less than optimally informative. In addition, the protein is covalently bound to the surface, preventing controlled binding and release of the protein.

Single molecule analysis of bound proteins has also been performed, e.g., using RNA polymerases that are coupled to a surface through an anti-HA antibody binding to an HA-tagged polymerase. See, e.g., Adelman et al. (2002) “Single Molecule Analysis of RNA Polymerase Elongation Reveals Uniform Kinetic Behavior” PNAS 99(21): 13538-13543. This polymerase was labeled at the N-terminus with a His-6 tag (for purification of the enzyme prior to attachment) and a C-terminal HA tag for binding to a surface. The anti-HA antibody was non-specifically adsorbed on the surface, which was additionally blocked with milk protein to reduce non-specific binding. However, such single label coupling methods can result in bound proteins being sub-optimally oriented relative to the surface, and the single attachment site is subject to the limitations of that particular attachment method (affinity, reversibility of binding, etc.). Surface effects can also reduce protein activity.

The present invention overcomes many of these limitations by insulating proteins to be bound to a surface from surface effects. Furthermore, the use of multiple attachments between the protein and the surface results in greater precision of orientation of bound protein, and adds controllability to the interaction of the protein on the surface. These and other features will be apparent upon review of the following.

### SUMMARY OF THE INVENTION

The invention includes enzymes that can be coupled to a surface, without substantial loss of enzymatic activity. Enzymes can be coupled to the surface through multiple surface coupling domains, which act in concert to increase binding affinity of the enzyme for the surface and to orient the enzyme relative to the surface. For example, the active site can be oriented distal to the surface, thereby making it accessible to an enzyme substrate. This orientation also tends to reduce surface denaturation effects in the region of the active site. In a related aspect, activity of the enzyme can be protected by making the coupling domains large, thereby serving to further insulate the active site from surface binding effects. Accordingly, isolated and/or recombinant enzymes comprising surface binding domains, surfaces with active enzymes bound to them, and methods of coupling enzymes to surfaces are all features of the invention.

Accordingly, in a first aspect, an isolated or recombinant enzyme comprising a plurality of artificial or recombinant surface coupling domains is provided. The enzyme, when coupled to a surface through the surface coupling domains, is enzymatically active. The enzyme can be any polypeptide that catalyzes a reaction, e.g., a polymerase, a DNA polymerase, an RNA polymerase, a reverse transcriptase, a helicase, a kinase, a caspase, a phosphatase, a terminal transferase, an endonuclease, an exonuclease, a dehydrogenase, a protease, a beta-lactamase, a beta-galactosidase, a luciferase, etc. For example, when the enzyme is a polymerase, the polymerase can be, e.g., any of a wide variety of polymerase enzymes, including for example, the Taq polymerases, exonuclease deficient Taq polymerases, E. coli DNA Polymerase 1, Klenow fragment, reverse transcriptases, Φ29 related polymerases including wild type Φ29 polymerase and derivatives of such polymerases such as exonuclease deficient forms, T7 DNA Polymerase, T5 DNA Polymerase, etc.

A variety of specific surface-coupleable Φ29 polymerases are exemplified herein, including those comprising a structural modification relative to the Φ29 DNA polymerase including, for example, those bearing mutations at or proximal to the enzyme's active site region, such as: a deletion of the residues 505-525, a deletion within residues 505-525, a K135A mutation, an E375H mutation, an E375S mutation, an E375K mutation, an E375R mutation, an E375A mutation, an E375Q mutation, an E375W mutation, an E375Y mutation, an E375F mutation, an L384R mutation, an E486A mutation, an E486D mutation, a K512A mutation, an N62D mutation, a D12A mutation, a T15I mutation, an E14I mutation, a D66A mutation, and/or combinations thereof. These polymerases comprise useful properties such as an ability to incorporate unnatural nucleotides, e.g., for the synthesis of nucleic acid polymer analogs, labeling nucleic acids during sequencing or amplification reactions, or the real-time monitoring of an incorporation event in the synthesis of nucleic acids, and/or decreased exonuclease activity.

Any of a variety of artificial surface coupling domains are included within the scope of the invention. The artificial surface coupling domain can simply be an in-frame fusion of a recombinant sequence to the enzyme, or it can be added post-translationally to the enzyme, e.g., chemically. Example coupling domains include any of: an added recombinant dimer of the whole or a portion or domain of the enzyme, a large extraneous polypeptide domain, a polyhistidine tag, a HIS-6 tag, a biotin, an avidin sequence, a GST sequence, a glutathione (e.g., chemically coupled to the polypeptide), a AviTag sequence, an S tag, a FLASH Tag, a SNAP-tag, an antibody, an oligonucleotide linker, an antibody domain, an antibody fragment, an antigen, a receptor, a receptor domain, a receptor fragment, a ligand, a dye, an acceptor, a quencher, and/or a combination thereof. The artificial surface coupling domains can include purification tags which are used, e.g., for enzyme purification, e.g., prior to binding of the enzyme to the surface (optionally through these same purification tags, or, optionally through different or additional surface binding domains).

In one aspect, the coupling domain is relatively large, e.g., at least 5 kDa in size. The relatively large size of the domain insulates the active site of the enzyme from surface effects, e.g., helping to prevent denaturation of the enzyme on the surface. The surface coupling domain can be e.g., at least 10 kDa, at least 20 kDa, at least 50 kDa, at least 100 kDa, or at least 1000 kDa or larger in size. These large coupling domains typically comprise polypeptide sequences that are sufficiently large to insulate the enzyme from the surface and can include any of those listed herein and optionally can include one or more additional sequences. For example, the large coupling domains can include a polypeptide sequence that includes a poly-His sequence fused to a large extraneous polypeptide sequence that is fused in frame to the enzyme sequence. The large coupling domain can also include two or more separate surface coupling elements, e.g., a poly-His sequence and a GST sequence.

In various embodiments, 1, 2, 3, 4, 5 . . . 10 or more coupling domains (which are optionally the same, or are optionally different domains) can be included in the enzyme (each of which can have 1, 2, 3 . . . or more different surface coupling elements). For example, in one specific embodiment, at least two different artificial coupling domains that are specifically bound by at least two different cognate binding components are included. In another example, at least three different artificial coupling domains that are specifically bound by at least three different cognate binding components are included.

Preferably, the artificial surface coupling domains are distal to an active site of the enzyme, and even more preferably are distal to the active site within the 3-dimensional structure of the enzyme. Without being bound to a particular theory of operation, it is believed that this acts to orient the enzyme active site away from the surface, making it accessible to enzyme ligands, and avoiding surface effects on the active site region of the enzyme. For example, when the active site is located within a C-terminal domain of the enzyme, the artificial surface coupling domain is located within an N-terminal domain of the enzyme, or vice versa. Enzyme orientation can be fixed relative to the surface through the use of multiple surface binding domains, by inhibiting enzyme rotation around surface coupling bonds. The use of multiple surface domains also increases binding affinity of the enzyme for a surface; for example, two surface coupling domains can have a higher binding affinity than binding of the enzyme to the surface through a single surface coupling domain (e.g., where the surface coupling domains have additive or synergistic effects on the overall binding affinity of the enzyme for the surface). The use of multiple domains can also facilitate purification and/or control release of the enzyme from a surface, by providing multiple different release mechanisms (e.g., coordinating metals from a nickel NTA binding domain in a first step, followed by other different release mechanisms such as heat, light, salt concentration, acid, base, etc., in a second controlled release step, depending on the nature of the additional coupling domains).

An advantage of the present system is that relatively high activity can be retained for the enzyme when bound to a surface. For example, the enzyme will typically have a k_{cat}/Kₘ (or Vₘₐₓ/Kₘ) that is at least 1% as high, or at least 10% as high as the enzyme in solution. Often the level will be at least 50% as high as the enzyme in solution, or 75% as high as the enzyme in solution, in some cases at least 90% as high, and even at least 95% as high or higher.

Accordingly, in a related aspect, the invention provides a surface comprising an active enzyme bound thereon. The enzyme is coupled to the surface through a plurality of artificial or recombinant surface coupling domains as discussed above, and typically displays a k_{cat}/Kₘ (or Vₘₐₓ/Kₘ) that is at least 10% as high as a corresponding active enzyme in solution.

A location of the enzyme on the surface is optionally fixed, providing a spatial address of the polymerase on the surface. The surface can be a planar surface, such as a chip, plate, slide, or the like, or can be a curved surface, e.g., as in a microwell plate, or can be a bead or other regular or irregular surface, such as porous surfaces or the like. The surface can include a polymer, a ceramic, glass, a bead, a microbead, a polymer bead, a glass bead, a well, a microwell, a slide, a grid, a rotor, a microchannel, or the like. The surface can be part of any existing instrumentation, e.g., in just one example, the surface can include, be within, or be proximal to a Zero Mode Wave Guide, which is used, e.g., for various optical analyses of single molecule reactions, such as sequencing applications that benefit from an active surface-bound DNA polymerase.

The surface may typically include a cognate binding moiety (a binding partner) that specifically binds to the surface coupling domain of the enzyme, e.g., the surface or the surface coupling domain can be any of those noted above. As noted, the surface coupling domains can comprise two or more different domains or binding elements and the immobilized component on the surface, correspondingly, can include at least two different complementary immobilized components. The different domains are optionally concurrently bound to the two different immobilized components; binding between different domains and immobilized components can occur, e.g., concurrently, simultaneously, or sequentially.

Methods of binding an enzyme to a surface are also provided. The methods include providing an isolated or recombinant enzyme that includes a plurality of artificial or recombinant surface coupling domains as noted above, along with a surface comprising a plurality of binding partners that specifically bind to the surface coupling domains. The enzyme is contacted with the surface, and the binding partners bind to the surface coupling domains, thereby binding the enzyme to the surface. This binding can be reversible, e.g., the enzyme can be released from the surface subsequent to binding the enzyme to the surface by disrupting binding between the binding partner and the coupling domain.

The surface coupling domain is optionally activatable, e.g., caged, e.g., photocaged. This facilitates controlled coupling to the surface. Contacting the enzyme to the surface can include activating (e.g., uncaging) the surface coupling domain. This activation can include, e.g., proteolysis, photolysis, chemical treatment of the enzyme or binding of an intermediate coupling moiety to the substrate.

### BRIEF DESCRIPTION OF THE DRAWINGS

FIG. 1 Panel A schematically illustrates interaction of a protein bearing a single His-6 tag with surface-immobilized nickel-NTA. Panel B depicts a plot of the progress of the interaction between a protein bearing a single His-6 tag and a BIAcore® sensor chip bearing immobilized nickel-NTA against time. Panel C schematically illustrates interaction of a protein bearing two His-6 tags with surface-immobilized nickel-NTA.

FIG. 2 schematically depicts a vector for expression of a recombinant Phi 29 DNA polymerase having three different surface coupling domains.

FIG. 3 Panels A-E schematically depict enzyme reactions in solution and on surfaces with and without various added surface insulating domains.

### DETAILED DESCRIPTION

### Overview

The ability to couple active enzymes to surfaces is useful in a variety of settings. For example, any enzyme activity can be measured in a solid phase format by binding the enzyme to a surface and performing the relevant assay. The ability to bind the enzyme to the surface has several advantages, including, but not limited to: the ability to purify, capture and assess enzyme reactions using a single substrate; the ability to re-use the enzyme by washing ligand and reagents off of the solid phase between uses; the ability to format bound enzymes into a spatially defined set of reactions by selecting where and how the enzyme is bound onto the solid phase, facilitating monitoring of the reactions (e.g., using available array detectors); the ability to perform and detect single-molecule reactions at defined sites on the substrate (thereby reducing reagent consumption); the ability to monitor multiple different enzymes on a single surface to provide a simple readout of multiple enzyme reactions at once, e.g., in biosensor applications, and many others.

Notwithstanding the foregoing advantages, in many, if not most cases, solid phase immobilization of enzymes, and particularly polymerase enzymes, can result in a significant diminution of enzyme activity, which are believed to result from surface effects on the enzyme, such as surface charge, relative hydrophobicity, steric interference from a nonoptimally oriented enzyme, or the like. While in many applications, this diminution in activity can be readily overcome by providing excess levels of enzyme, and thereby flooding out any reduction in activity, such remedial measures may not be practicable in all circumstances. For example, excess enzyme concentrations are not a viable option in applications that necessarily rely on very low concentrations of the enzyme, e.g., single molecule detection based analyses.

As discussed, there are several problems in the prior art associated with coupling proteins to surfaces. These include protein denaturation on the surface (e.g., due to hydrophobic or hydrophilic properties of the surface, or even simply steric effects between the protein and the surface); a lack of specific orientation of bound proteins, providing inconsistent properties between bound proteins, depending on orientation of individual proteins relative to the substrate (making single molecule readouts difficult to implement in the prior art); a lack of sufficient affinity between the protein and the surface for non-covalent linkages, a lack of controllability of binding of the protein to a surface, and many others.

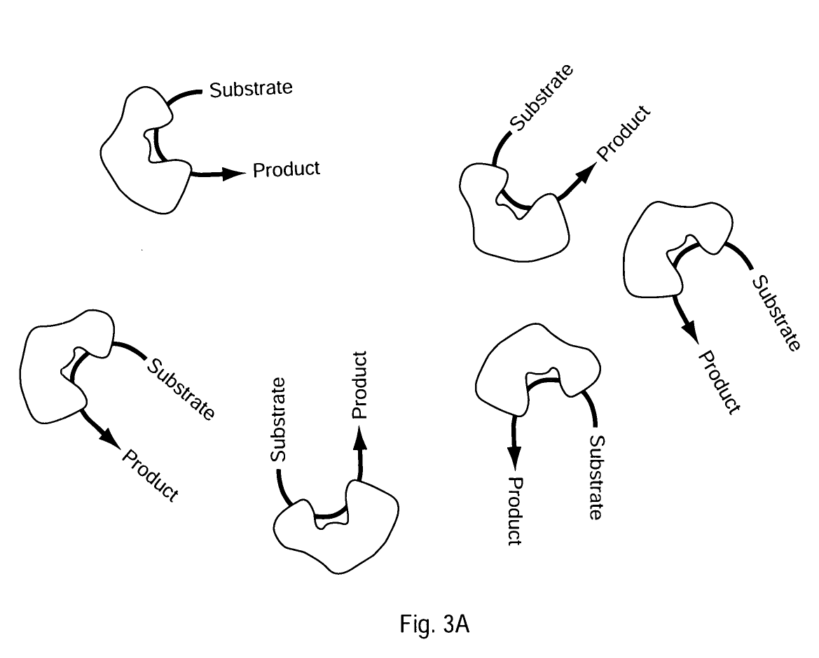

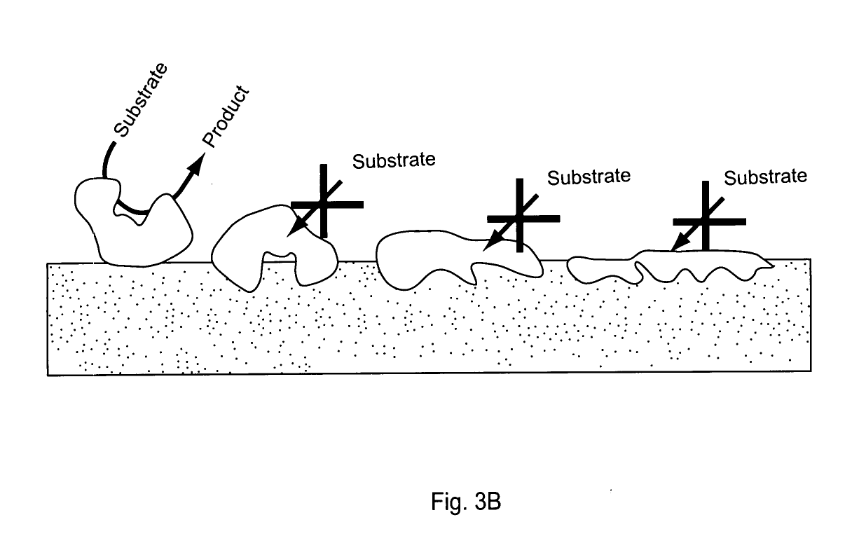

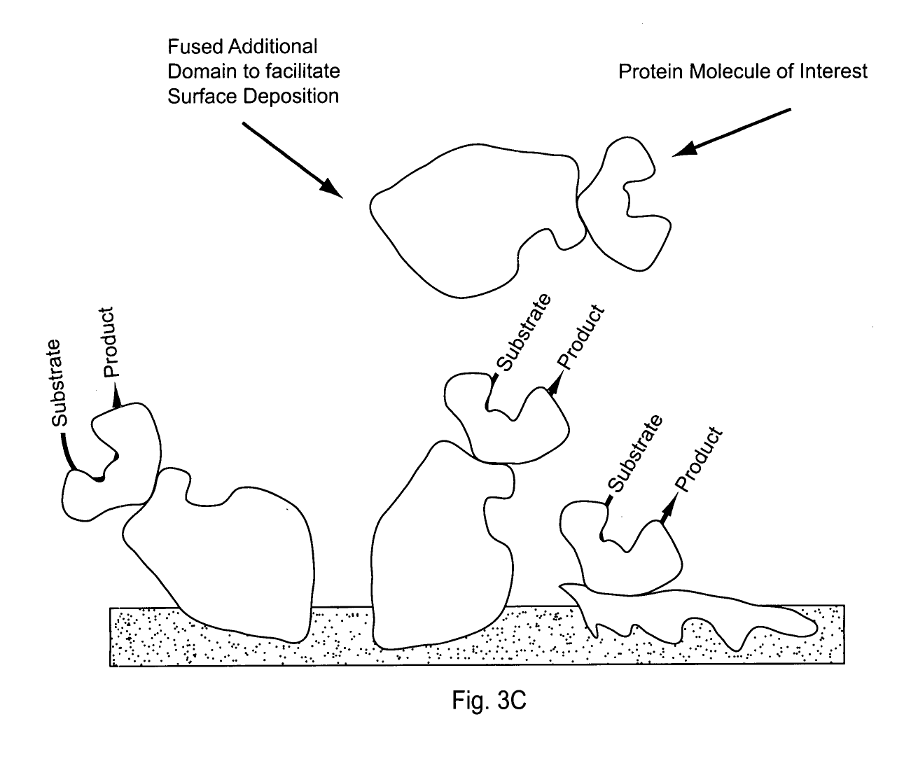

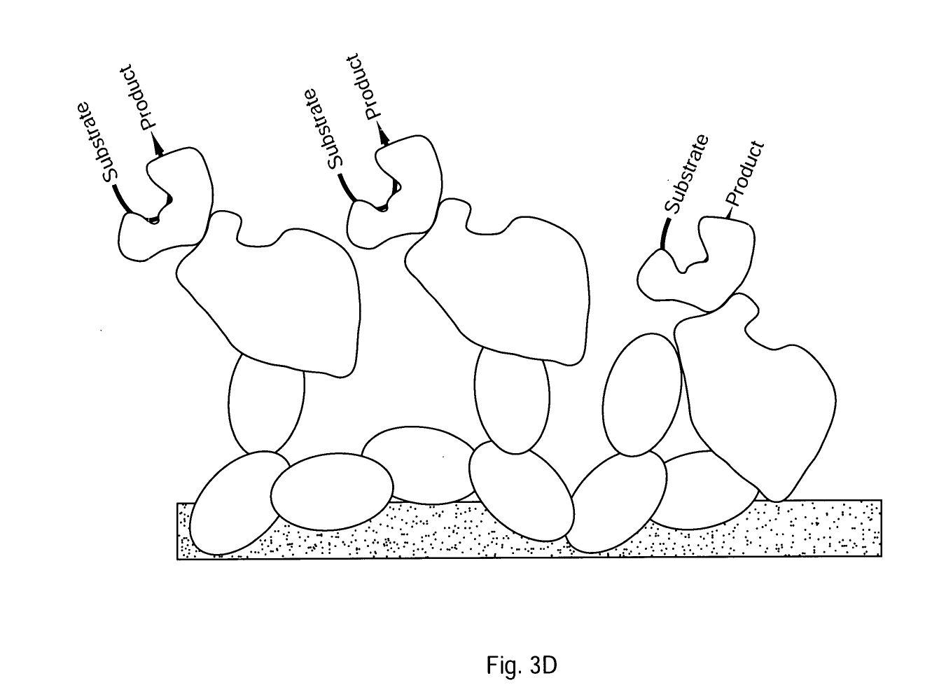

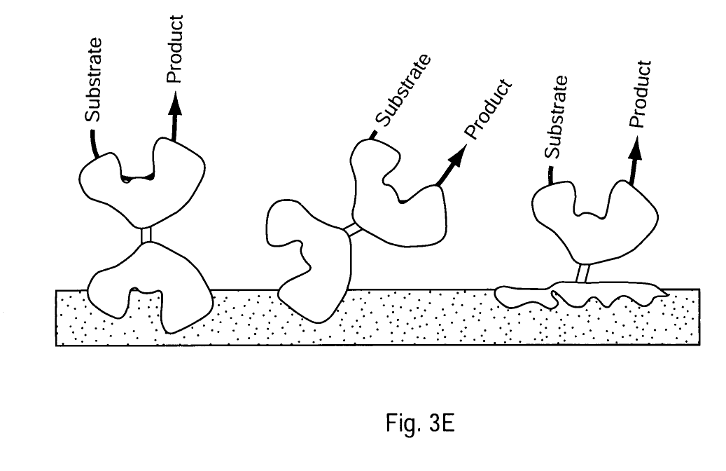

This is schematically illustrated in FIGS. 3A and 3B. FIG. 3A schematically shows a typical enzyme reaction in which an enzyme converts a substrate into a product. FIG. 3B schematically depicts denaturation of the enzyme when bound to a surface and/or steric blocking of the enzyme's active site by the surface, resulting in reduced enzymatic activity (the enzyme can't access the substrate and/or convert it to product).

The present invention overcomes these difficulties by various interrelated approaches. First, to combat surface effects, the protein (e.g., enzyme) can be coupled to a relatively large insulating linker moiety such as a large protein domain (at least 5 kDa, and preferably larger) that insulates the protein from the surface. Second, two or more surface binding elements can be used to specifically orient the protein relative to the surface (binding of the overall protein to the surface at two or more sites inhibits rotation of the protein and tends to orient the protein relative to the surface). Third, the insulating moiety and/or the surface binding elements are placed distal to the biologically relevant portion of the protein, e.g., in the case of enzymes, the active site.

Embodiments of these strategies are schematically illustrated in FIGS. 3C-3E. As shown in FIG. 3C, a large domain is fused to the enzyme to produce a fusion enzyme. The large domain is coupled to the surface, insulating the enzymatic portion of the fusion enzyme from the surface, making the enzymatically active portion of the fusion enzyme available for substrate binding and conversion to product. As schematically shown in FIG. 3D, the large domain can include features that tether the fusion enzyme to the surface, e.g., domains that are recognized by surface bound antibodies or antibody components. FIG. 3E schematically shows an example fusion enzyme that comprises a fusion of two monomer forms of an enzyme to form a dimer. One of the dimer domains insulates the other domain from the surface upon being bound to the surface. Further, by selecting orientation of the enzyme domains of the dimer, at least one of the active sites will be positioned away from the surface upon binding of the other domain.

Accordingly, an advantageous feature of the invention is that enzymes can be coupled to a surface using large insulating domains and/or multiple coupling sites to the surface, without substantial loss of enzymatic activity. Single molecule enzyme readouts (or a small number of grouped molecule readouts) can be achieved, with reasonable consistency between individual surface-bound enzyme molecules, facilitating a variety of extremely small volume reactions.

Accordingly, isolated and/or recombinant enzymes comprising surface binding domains, surfaces with active enzymes bound to them, and methods of coupling enzymes to surfaces are all features of the invention.

### Enzymes

An enzyme is a molecule that catalyzes a reaction of interest. Typically, the enzyme is or comprises a polypeptide. A variety of polypeptide enzymes are known, e.g., polymerases (e.g., DNA polymerases, RNA polymerases, reverse transcriptases, terminal transferases), helicases, kinases, caspases, phosphatases, terminal transferases, endonucleases, exonucleases, dehydrogenases, proteases, beta-lactamase, beta-galactosidases, luciferases, etc.

Known polypeptide enzymes have been grouped into six classes (and a number of subclasses and sub-subclasses) under the Enzyme Commission classification scheme (see, e.g. the Nomenclature Committee of the International Union of Biochemistry and Molecular Biology enzyme nomenclature pages, on the world wide web at www(dot)chem(dot)qmul(dot)ac(dot)uk/iubmb/enzyme), namely, oxidoreductase, transferase, hydrolase, lyase, ligase, and isomerase. Any of these general classes of enzymes can be bound to a surface using the various strategies herein.

Accordingly, the enzyme to be coupled to a surface can be essentially any enzyme. For example, the enzyme can be an oxidoreductase from any one of EC subclasses 1.1-1.21 or 1.97, a transferase from any one of EC subclasses 2.1-2.9 (e.g., a nucleotidyltransferase from sub-subclass 2.7.7, e.g., a DNA-directed DNA polymerase from 2.7.7.7), a hydrolase from any one of EC subclasses 3.1-3.13, a lyase from any one of EC subclasses 4.1-4.6 or 4.99, an isomerase from any one of EC subclasses 5.1-5.5 or 5.99, or a ligase from any one of EC subclasses 6.1-6.6.

In a most preferred aspect, nucleic acid enzymes, such as polymerases, ligases, nucleases, and the like, are preferred classes of enzymes, with polymerases being most preferred. Notwithstanding the foregoing, a wide variety of pharmaceutically relevant enzyme types are of significant interest in conjunction with the present invention, as their immobilization provides readily analyzable formats for screening for inhibitors, modulators and effectors to such enzyme systems. Such enzymes include kinases, phosphatases, proteases, as well as the aforementioned nucleic acid enzymes.

DNA Polymerases

One preferred class of enzymes of the invention that can be fixed to a surface are DNA polymerases. For example, DNA template-dependent DNA polymerases have relatively recently been classified into six main groups based upon various phylogenetic relationships, e.g., with E. coli Pol I (class A), E. coli Pol II (class B), E. coli Pol III (class C), Euryarchaeotic Pol II (class D), human Pol beta (class X), and E. coli UmuC/DinB and eukaryotic RAD30/xeroderma pigmentosum variant (class Y). For a review of recent nomenclature, see, e.g., Burgers et al. (2001) “Eukaryotic DNA polymerases: proposal for a revised nomenclature” J Biol Chem. 276(47):43487-90. For a review of polymerases, see, e.g., Hübscher et al. (2002) EUKARYOTIC DNA POLYMERASES Annual Review of Biochemistry Vol. 71: 133-163; Alba (2001) “Protein Family Review: Replicative DNA Polymerases” Genome Biology 2(1):reviews 3002.1-3002.4; and Steitz (1999) “DNA polymerases: structural diversity and common mechanisms” J Biol Chem 274:17395-17398. The basic mechanisms of action for many polymerases have been determined. The sequences of literally hundreds of polymerases are publicly available, and the structures for many of these have been determined, or can be inferred based upon similarity to solved crystal structures for homologous polymerases. Polymerases like those set forth herein are particularly vulnerable to diminution of activity upon immobilization upon a solid support, and thus would greatly benefit from the present invention.

For example, when the enzyme is a DNA polymerase, the polymerase can be, e.g., any of the Taq polymerases, exonuclease deficient Taq polymerases, E. coli DNA Polymerase 1, Klenow fragment, reverse transcriptases, Φ29 related polymerases including wild type Φ29 polymerase and derivatives of such polymerases such as exonuclease deficient forms, T7 DNA Polymerase, T5 DNA Polymerase, etc. Further details regarding DNA polymerases, including DNA polymerases that comprise mutations that improve the ability of the polymerase to incorporate unnatural nucleotides (useful in a variety of sequencing and labeling applications), are found in Attorney Docket number 105-001310US “POLYMERASES FOR NUCLEOTIDE ANALOGUE INCORPORATION” by Hanzel et al., co-filed herewith and incorporated herein by reference in its entirety, and in U.S. patent application 60/753,670 entitled “POLYMERASES FOR NUCLEOTIDE ANALOGUE INCORPORATION” by Hanzel et al., filed Dec. 22, 2005, also incorporated herein by reference in its entirety.

### Coupling Domains

An artificial surface coupling domain is a moiety that is heterologous to the protein (e.g., enzyme) of interest, and that is capable of binding to a binding partner that is coupled or bound to (and/or integral with) a surface. For convenience, the coupling domain will often be expressed as a fusion domain of the overall protein, e.g., as a conventional in-frame fusion of a surface coupling domain polypeptide sequence with the active enzyme (e.g., a poly-His tag fused in frame to an active enzyme sequence). However, coupling domains can also be added chemically to the protein, e.g., by using an available amino acid residue of the enzyme, or by incorporating an amino acid into the protein that provides a suitable attachment site for the coupling domain. Suitable residues of the enzyme can include, e.g., histidine, cysteine or serine residues (providing for N, S or O linked coupling reactions), or glycosylation sites (e.g., the binding partner can be an antibody or receptor that binds to a polysaccharide glycosylation structure of the coupling domain). Unnatural amino acids that comprise unique reactive sites can also be added to the enzyme, e.g., by expressing the enzyme in a system that comprises an orthogonal tRNA and an orthogonal synthetase that incorporate the unnatural amino acid during polypeptide synthesis in response to a selector codon.

A single type of coupling domain, or more than one type can be included. 1, 2, 3, 4, 5 . . . 10 or more coupling domains (which are optionally the same, or are optionally different domains) can be included in the enzyme. Furthermore each domain can have 1, 2, 3, 4, 5 . . . 10 or more different surface coupling elements. For example, a large surface coupling domain, e.g., a domain that includes a polypeptide domain of at least 5 kDa, and preferably larger, can optionally includes multiple surface coupling elements. In contrast, a small coupling domain such as a poly-His domain optionally includes a single coupling element (e.g., a poly-His sequence). Thus, large coupling domains can include multiple coupling elements, and enzymes of the invention can include one or more large coupling domains, and/or two or more coupling domains in general.

Types of Coupling Domains/Elements

Example coupling domains (which can be coupled to the protein/enzyme, e.g., as an in frame fusion domain or as a chemically coupled domain) include any of: an added recombinant dimer enzyme or portion or domain of the enzyme, a large extraneous polypeptide domain, a polyhistidine tag, a HIS-6 tag, a biotin, an avidin sequence, a GST sequence, a glutathione, a AviTag sequence, an S tag, an antibody, an antibody domain, an antibody fragment, an antigen, a receptor, a receptor domain, a receptor fragment, a ligand, a dye, an acceptor, a quencher, and/or a combination thereof. The artificial surface coupling domains can include purification tags which are used, e.g., for enzyme purification, e.g., prior to binding of the enzyme to the surface (optionally through these same purification tags, or, optionally through different or additional surface binding domains), or concomitant with binding to the surface (e.g., the surface is optionally used for affinity capture of the enzyme).

A large number of tags are known in the art and can be adapted to the practice of the present invention by being incorporated as coupling domains/elements. For example, see, e.g.: Nilsson et al. (1997) “Affinity fusion strategies for detection, purification, and immobilization of recombinant proteins” Protein Expression and Purification 11: 1-16, Terpe et al. (2003) “Overview of tag protein fusions: From molecular and biochemical fundamentals to commercial systems” Applied Microbiology and Biotechnology 60:523-533, and references therein. Tags that can be used to couple the enzyme to the surface through binding to an immobilized binding partner include, but are not limited to, a polyhistidine tag (e.g., a His-6, His-8, or His-10 tag) that binds immobilized divalent cations (e.g., Ni²⁺), a biotin moiety (e.g., on an in vivo biotinylated polypeptide sequence) that binds immobilized avidin, a GST (glutathione S-transferase) sequence that binds immobilized glutathione, an S tag that binds immobilized S protein, an antigen that binds an immobilized antibody or domain or fragment thereof (including, e.g., T7, myc, FLAG, and B tags that bind corresponding antibodies), a FLASH Tag (a high affinity tag that couples to specific arsenic based moieties), a receptor or receptor domain that binds an immobilized ligand (or vice versa), protein A or a derivative thereof (e.g., Z) that binds immobilized IgG, synthetic binding peptides (see, e.g., U.S. Pat. No. 5,491,074), maltose-binding protein (MBP) that binds immobilized amylose, an albumin-binding protein that binds immobilized albumin, a chitin binding domain that binds immobilized chitin, a calmodulin binding peptide that binds immobilized calmodulin, and a cellulose binding domain that binds immobilized cellulose. Another exemplary tag that can be used to couple the enzyme to the surface is a SNAP-tag, commercially available from Covalys (www(dot)covalys(dot)com). The SNAP-tag is an approximately 20 kDa version of a protein O⁶-alkylguanine-DNA alkyltransferase which has a single reactive cysteine with a very high affinity for guanines alkylated at the O⁶-position. The alkyl group, including any immobilization moiety attached to the alkyl group (e.g., a surface-immobilized alkyl group), is transferred covalently from the guanine to the cysteine in the alkyltransferase protein.

One or more specific protease recognition sites are optionally included in a coupling domain, for example, between adjacent tags or between a tag and the enzyme. Example specific proteases include, but are not limited to, thrombin, enterokinase, factor Xa, TEV protease, and HRV 3C protease. Similarly, an intein sequence can be incorporated into a coupling domain (e.g., an intein that undergoes specific self cleavage in the presence of free thiols). Such protease cleavage sites and/or inteins are optionally used to remove a tag used for purification of the enzyme and/or for releasing the enzyme from the surface.

Large Coupling Domains

In one aspect, the coupling domain is relatively large, e.g., at least 5 kDa in size. These large domains can be added to the protein recombinantly (e.g., as in-frame fusions) or post-translationally (e.g., chemically). The relatively large size of the domain insulates the active site of the enzyme from surface effects, e.g., helping to prevent denaturation of the enzyme on the surface. The surface coupling domain can be e.g., at least 5 kDa, at least 10 kDa, at least 20 kDa, at least 50 kDa, at least 100 kDa, at least 1000 kDa or larger in size. These large coupling domains can include any of those listed herein and optionally can include one or more additional sequences. For example, the domains can include a large polypeptide sequence. The polypeptide sequence can, but does not necessarily, include coupling elements, e.g., fused to the large polypeptide sequence. Thus, for example, a large extraneous surface insulating polypeptide sequence can be fused in frame to the enzyme sequence and a coupling element such as a poly-His sequence. The large coupling domain can also include two or more separate surface coupling elements, e.g., a poly-His sequence and a GST sequence, e.g., in addition to a large polypeptide sequence that insulates enzymatic domains from the surface.

Examples of large coupling domains can include, e.g., one or more polypeptide sequence. For example, a sequence that is inactive relative to the enzyme of interest (e.g., has little or no effect on enzymatic activity) can be used. Such sequences include polypeptide chains of known polypeptides, random sequences, or sequences selected by the user. Sequences that are likely to disrupt folding of the enzyme are typically avoided, e.g., the large coupling domain is typically selected to avoid charged or reactive residues proximal to the enzyme domain of a fusion protein (though the large domain can present charged or reactive residues distal to the enzyme, e.g., to interact with the surface or binding partner). The large coupling domain optionally includes a polypeptide sequence that improves solubility of the coupling domain-enzyme fusion protein, for example, MBP, thioredoxin, or NusA (N utilization substance A).

The large coupling domain can fold upon translation into a defined structure, e.g., as a protein or protein domain. A wide variety of structurally discrete domains are known in the literature and can be used as large coupling domains. The NCBI, GeneBank and others provide extensive lists of known polypeptide sequences that can be used, in whole or in part, as large coupling domains. Furthermore, random sequences, or sequences designed by the user to have appropriate properties (e.g., by including coupling elements, charged features proximal to oppositely charged surface features, regions of secondary structure such as helixes, turns, hydrophobic or hydrophilic domains, etc.) can be used. These structures can be partially or fully denatured upon binding to the surface, insulating or “cushioning” the active enzyme from the surface.

Fusion Proteins

The recombinant construction of fusion proteins is generally well known and can be applied to the present invention to incorporate coupling domains or elements. In brief, a nucleic acid that encodes the coupling domain or element is fused in frame to a nucleic acid encoding the enzyme of interest. The resulting fusion nucleic acid is expressed (in vitro or in vivo) and the expressed fusion protein is isolated, e.g., by standard methods and/or by binding coupling elements, e.g., comprising purification tags, to surfaces. Coupling domains or elements are typically fused N-terminal and/or C-terminal to the enzyme, but are optionally internal to the enzyme (e.g., incorporated into a surface loop or the like) where such incorporation does not interfere with function of the enzyme or domain).

References that discuss recombinant methods that can be used to construct fusion nucleic acids and to create fusion proteins include Sambrook et al., Molecular Cloning—A Laboratory Manual (3rd Ed.), Vol. 1-3, Cold Spring Harbor Laboratory, Cold Spring Harbor, N.Y., 2000 (“Sambrook”); Current Protocols in Molecular Biology, F. M. Ausubel et al., eds., Current Protocols, a joint venture between Greene Publishing Associates, Inc. and John Wiley & Sons, Inc., (supplemented through 2005) (“Ausubel”)) and PCR Protocols A Guide to Methods and Applications (Innis et al. eds) Academic Press Inc. San Diego, Calif. (1990) (Innis).

In addition, a plethora of kits are commercially available for cloning, recombinant expression and purification of plasmids or other relevant nucleic acids from cells, (see, e.g., EasyPrep™, FlexiPrep™, both from Pharmacia Biotech; StrataClean™, from Stratagene; and, QIAprep™ from Qiagen). Any isolated and/or purified nucleic acid can be further manipulated to produce other nucleic acids, used to transfect cells, incorporated into related vectors to infect organisms for expression, and/or the like. Typical cloning vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or both. See, Giliman & Smith, Gene 8:81 (1979); Roberts, et al., Nature, 328:731 (1987); Schneider, B., et al., Protein Expr. Purif. 6435:10 (1995); Ausubel, Sambrook, Berger (above). A catalogue of Bacteria and Bacteriophages useful for cloning is provided, e.g., by the ATCC, e.g., The ATCC Catalogue of Bacteria and Bacteriophage published yearly by the ATCC. Additional basic procedures for sequencing, cloning and other aspects of molecular biology and underlying theoretical considerations are also found in Watson et al. (1992) Recombinant DNA Second Edition, Scientific American Books, NY.

Other useful references, e.g. for cell isolation and culture (e.g., for subsequent nucleic acid isolation and fusion protein expression) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Atlas and Parks (eds) The Handbook of Microbiological Media (1993) CRC Press, Boca Raton, Fla.

In addition, essentially any fusion nucleic acid can be custom or standard ordered from any of a variety of commercial sources, such as Operon Technologies Inc. (Alameda, Calif.).

A variety of protein isolation and detection methods are known and can be used to isolate enzymes, e.g., from recombinant cultures of cells expressing fusion protein enzymes of the invention. A variety of protein isolation and detection methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; Walker (2002) Protein Protocols on CD-ROM, version 2.0 Humana Press, NJ; Current Protocols in Protein Science, John E. Coligan et al., eds., Current Protocols, a joint venture between Greene Publishing Associates, Inc. and John Wiley & Sons, Inc., (supplemented through 2005); and the references cited therein. Additional details regarding protein purification and detection methods can be found in Satinder Ahuja ed., Handbook of Bioseparations, Academic Press (2000).

Adding Coupling Domains Chemically

In addition to the convenient recombinant expression of fusion proteins comprising coupling domains, the coupling domains can also alternatively or additionally be coupled to the enzyme chemically. For example, N, S or O containing residues of the enzyme (or added recombinantly to the enzyme) can be coupled through standard chemical methods to coupling domains that comprise groups that bind these residues.

In addition, systems of orthogonal components are available that can incorporate any of a variety of chemically reactive unnatural amino acids into a recombinant protein. In brief, a cell or other translation system is constructed that includes an orthogonal tRNA (“OtRNA”; a tRNA not recognized by the cell's endogenous translation machinery, such as an amber or 4-base tRNA) and an orthogonal tRNA synthetase (“ORS”; this is a synthetase that does not aminoacylate any endogenous tRNA of the cell, but which can aminoacylate the OtRNA in response to a selector codon). A nucleic acid encoding the enzyme is constructed to include a selector codon at a selected that is specifically recognized by the OtRNA. The ORS specifically incorporates an unnatural amino acid with a desired chemical functionality at one or more selected site(s) (e.g., distal to the active site). This chemical functional group can be unique as compared to those ordinarily found on amino acids, e.g., that incorporate keto or other functionalities. These are coupled to the coupling domains through appropriate chemical linkages.

Further information on orthogonal systems can be found, e.g., in Wang et al., (2001), Science 292:498-500; Chin et al., (2002) Journal of the American Chemical Society 124:9026-9027; Chin and Schultz, (2002), ChemBioChem 11:1135-1137; Chin, et al., (2002), PNAS United States of America 99:11020-11024; and Wang and Schultz, (2002), Chem. Comm., 1-10. See also, International Publications WO 2002/086075, entitled “METHODS AND COMPOSITIONS FOR THE PRODUCTION OF ORTHOGONAL tRNA AMINOACYL-tRNA SYNTHETASE PAIRS;” WO 2002/085923, entitled “IN VIVO INCORPORATION OF UNNATURAL AMINO ACIDS;” WO 2004/094593, entitled “EXPANDING THE EUKARYOTIC GENETIC CODE;” WO 2005/019415, filed Jul. 7, 2004; WO 2005/007870, filed Jul. 7, 2004; and WO 2005/007624, filed Jul. 7, 2004.

Orientation Properties

Preferably, the artificial surface coupling domains are distal to an active site of the enzyme, and more preferably, distal in the context of the 3-dimensional structure of the enzyme. By “distal to an active site”, in the context of the present invention, is meant a position in the enzyme structure that is closer to a particular point in the space occupied by the enzyme (e.g., 3-dimensional space) than it is to an average location of the active site of the enzyme, where the ‘particular point’ is the point in the enzyme structure that is furthest from the average location of the active site. Without being bound to any particular theory of operation, it is believed that this tends to orient the enzyme active site away from the surface, making it accessible to enzyme substrates, and avoiding surface effects on the active site region of the enzyme. For example, when the active site is located toward the C-terminal domain of the enzyme, the artificial surface coupling domain will generally be located more toward the N-terminal domain of the enzyme, or vice versa. Of course, in preferred aspects, the relative positioning of the artificial surface coupling domain to the active site is defined in the context of the 3-dimensional structure of the enzyme, which may or may not positionally map to the primary structure of the enzyme, e.g., both active site and coupling domain may be within the C terminal region in the primary structure of the protein, but still be distal from each other when examined with respect to the secondary or tertiary structure of the protein. Enzyme orientation can be fixed relative to the surface through the use of multiple surface binding domains or elements, by inhibiting enzyme rotation around surface coupling bonds. The use of multiple surface domains also increases binding affinity of the enzyme for a surface; for example, two surface coupling domains can have a higher binding affinity than binding of the enzyme to the surface through a single surface coupling domain (e.g., where the surface coupling domains have additive or synergistic effects on the overall binding affinity of the enzyme for the surface). The use of multiple domains can also facilitate purification and/or control release of the enzyme from a surface, by providing multiple different release mechanisms (e.g., coordinating metals from a nickel NTA binding domain in a first step, followed by other different release mechanisms such as heat, light, salt concentration, acid, base, site-specific protease treatment, binding competition, etc., in a second controlled release step, depending on the nature of the additional coupling domains).

Controllable Coupling

In many solid-phase applications, it is useful to control coupling of the surface coupling domain and the binding partner. For example, standard chip masking strategies can be used to selectively block or expose surface bound binding partners to one or more un-blocking action (exposure to light, heat, chemicals, pH, protein blocking agents, etc.). The coupling domain can similarly be blocked until it is desirable to couple it to the binding partner. This blocking/unblocking approach can be used to create complex arrays of proteins (e.g., enzymes) coupled to the surface. This is useful in array-based applications, e.g., where the activity of the enzyme is monitored at selected sites on the array, e.g., using standard array detectors.

Thus, coupling of the surface coupling domain to the surface is optionally controlled by caging the surface coupling domain and/or its binding partner. The surface coupling domain or its partner can be caged, for example, by attachment of at least one photolabile caging group to the domain or partner; the presence of the caging group prevents the interaction of the surface coupling domain with its binding partner, while removal of the caging group by exposure to light of an appropriate wavelength permits the interaction to occur. The photolabile caging group can be, e.g., a relatively small moiety such as carboxyl nitrobenzyl, 2-nitrobenzyl, nitroindoline, hydroxyphenacyl, DMNPE, or the like, or it can be, e.g., a relatively bulky group (e.g. a macromolecule, a protein) covalently attached to the molecule by a photolabile linker (e.g., a polypeptide linker comprising a 2-nitrophenyl glycine residue). Other caging groups can be removed from a molecule, or their interference with the molecule's activity can be otherwise reversed or reduced, by exposure to an appropriate type of uncaging energy and/or exposure to an uncaging chemical, enzyme, or the like.

A large number of caging groups, and a number of reactive compounds that can be used to covalently attach caging groups to other molecules, are well known in the art. Examples of photolabile caging groups include, but are not limited to: nitroindolines; N-acyl-7-nitroindolines; phenacyls; hydroxyphenacyl; brominated 7-hydroxycoumarin-4-ylmethyls (e.g., Bhc); benzoin esters; dimethoxybenzoin; meta-phenols; 2-nitrobenzyl; 1-(4,5-dimethoxy-2-nitrophenyl)ethyl (DMNPE); 4,5-dimethoxy-2-nitrobenzyl (DMNB); alpha-carboxy-2-nitrobenzyl (CNB); 1-(2-nitrophenyl)ethyl (NPE); 5-carboxymethoxy-2-nitrobenzyl (CMNB); (5-carboxymethoxy-2-nitrobenzyl)oxy) carbonyl; (4,5-dimethoxy-2-nitrobenzyl)oxy) carbonyl; desoxybenzoinyl; and the like. See, e.g., U.S. Pat. No. 5,635,608 to Haugland and Gee (Jun. 3, 1997) entitled “α-carboxy caged compounds”; Neuro 19, 465 (1997); J Physiol 508.3, 801 (1998); Proc Natl Acad Sci USA 1988 September, 85(17):6571-5; J Biol Chem 1997 Feb. 14, 272(7):4172-8; Neuron 20, 619-624, 1998; Nature Genetics, vol. 28:2001:317-325; Nature, vol. 392, 1998:936-941; Pan, P., and Bayley, H. “Caged cysteine and thiophosphoryl peptides” FEBS Letters 405:81-85 (1997); Pettit et al. (1997) “Chemical two-photon uncaging: a novel approach to mapping glutamate receptors” Neuron 19:465-471; Furuta et al. (1999) “Brominated 7-hydroxycoumarin-4-ylmethyls: novel photolabile protecting groups with biologically useful cross-sections for two photon photolysis” Proc. Natl. Acad. Sci. 96(4):1193-1200; Zou et al. “Catalytic subunit of protein kinase A caged at the activating phosphothreonine” J. Amer. Chem. Soc. (2002) 124:8220-8229; Zou et al. “Caged Thiophosphotyrosine Peptides” Angew. Chem. Int. Ed. (2001) 40:3049-3051; Conrad I I et al. “p-Hydroxyphenacyl Phototriggers: The reactive Excited State of Phosphate Photorelease” J. Am. Chem. Soc. (2000) 122:9346-9347; Conrad I I et al. “New Phototriggers 10: Extending the π,π* Absorption to Release Peptides in Biological Media” Org. Lett. (2000) 2:1545-1547; Givens et al. “A New Phototriggers 9: p-Hydroxyphenacyl as a C-Terminus Photoremovable Protecting Group for Oligopeptides” J. Am. Chem. Soc. (2000) 122:2687-2697; Bishop et al. “40-Aminomethyl-2,20-bipyridyl-4-carboxylic Acid (Abc) and Related Derivatives: Novel Bipyridine Amino Acids for the Solid-Phase Incorporation of a Metal Coordination Site Within a Peptide Backbone” Tetrahedron (2000) 56:4629-4638; Ching et al. “Polymers As Surface-Based Tethers with Photolytic triggers Enabling Laser-Induced Release/Desorption of Covalently Bound Molecules” Bioconjugate Chemistry (1996) 7:525-8; BioProbes Handbook, 2002 from Molecular Probes, Inc.; and Handbook of Fluorescent Probes and Research Products, Ninth Edition or Web Edition, from Molecular Probes, Inc, as well as the references below.

Caged polymerases (e.g., caged surface coupling domains and/or binding partners) can be produced, e.g., by reacting a polypeptide with a caging compound or by incorporating a caged amino acid during synthesis of a polypeptide. See, e.g., U.S. Pat. No. 5,998,580 to Fay et al. (Dec. 7, 1999) entitled “Photosensitive caged macromolecules”; Kossel et al. (2001) PNAS 98:14702-14707; Trends Plant Sci (1999) 4:330-334; PNAS (1998) 95:1568-1573; J Am Chem Soc (2002) 124:8220-8229; Pharmacology & Therapeutics (2001) 91:85-92; and Angew Chem Int Ed Engl (2001) 40:3049-3051. A polypeptide can be reacted with a caged biotin (see, e.g., Pirrung and Huang (1996) “A general method for the spatially defined immobilization of biomolecules on glass surfaces using ‘caged’ biotin” Bioconiug Chem. 7:317-21). As another example, a photolabile polypeptide linker (e.g., comprising a photolabile amino acid such as that described in U.S. Pat. No. 5,998,580, supra) can be used to link a bulky caging group (e.g., another polypeptide that blocks the interaction between the surface coupling domain and its binding partner) to the surface coupling domain or partner.

Useful site(s) of attachment of caging groups to a given molecule can be determined by techniques known in the art. For example, a surface coupling domain can be reacted with a caging compound. The resulting caged surface coupling domain can then be tested to determine if its interaction with its binding partner is sufficiently blocked. As another example, for a polypeptide surface coupling domain, amino acid residues located at the surface coupling domain-partner binding interface can be identified by routine techniques such as scanning mutagenesis, sequence comparisons and site-directed mutagenesis, or the like. Such residues in the coupling domain can then be caged, and the activity of the caged surface coupling domain can be assayed to determine the efficacy of caging.

Appropriate methods for uncaging caged molecules are also known in the art. For example, appropriate wavelengths of light for removing many photolabile groups have been described; e.g., 300-360 nm for 2-nitrobenzyl, 350 nm for benzoin esters, and 740 nm for brominated 7-hydroxycoumarin-4-ylmethyls (see, e.g., references herein). Conditions for uncaging any caged molecule (e.g., the optimal wavelength for removing a photolabile caging group) can be determined according to methods well known in the art. Instrumentation and devices for delivering uncaging light are likewise known; for example, well-known and useful light sources include e.g., a lamp or a laser.

Properties of Bound Enzymes/Determining Kinetic Parameters

The bound enzyme will typically have a k_{cat}/Kₘ (or Vₘₐₓ/Kₘ) that is at least 10% as high as the enzyme in solution. Often the level will be at least 50% as high as the enzyme in solution, or at least 75% as high as the enzyme in solution, at least 90% as high, or in some cases, at least 95% as high as the enzyme in solution, or higher.

The enzymes of the invention can be screened (in solution or on a solid phase) or otherwise tested to determine whether and to what degree the enzyme is active. For example, k_{cat}, Kₘ, Vₘₐₓ, or k_{cat}/Kₘ of the enzyme can be determined.

For example, as is well-known in the art, for enzymes obeying simple Michaelis-Menten kinetics, kinetic parameters are readily derived from rates of catalysis measured at different substrate concentrations. The Michaelis-Menten equation, V=Vₘₐₓ[S]([S]+Kₘ)⁻¹, relates the concentration of uncombined substrate ([S], approximated by the total substrate concentration), the maximal rate (Vₘₐₓ, attained when the enzyme is saturated with substrate), and the Michaelis constant (Kₘ, equal to the substrate concentration at which the reaction rate is half of its maximal value), to the reaction rate (V).

For many enzymes, Kₘ is equal to the dissociation constant of the enzyme-substrate complex and is thus a measure of the strength of the enzyme-substrate complex. For such an enzyme, in a comparison of Kₘ's, a lower Kₘ represents a complex with stronger binding, while a higher Kₘ represents a complex with weaker binding. The ratio k_{cat}/Kₘ, sometimes called the specificity constant, represents the apparent rate constant for combination of substrate with free enzyme. The larger the specificity constant, the more efficient the enzyme is in binding the substrate and converting it to product.

The k_{cat} (also called the turnover number of the enzyme) can be determined if the total enzyme concentration ([E_{T}], i.e., the concentration of active sites) is known, since Vₘₐₓ=k_{cat}[E_{T}]. For situations in which the total enzyme concentration is difficult to measure, the ratio Vₘₐₓ/Kₘ is often used instead as a measure of efficiency. Kₘ and Vₘₐₓ can be determined, for example, from a Lineweaver-Burk plot of 1/V against 1/[S], where the y intercept represents 1/Vₘₐₓ, the x intercept −1/Kₘ, and the slope Kₘ/Vₘₐₓ, or from an Eadie-Hofstee plot of V against V/[S], where the y intercept represents Vₘₐₓ, the x intercept Vₘₐₓ/Kₘ, and the slope −Kₘ. Software packages such as KinetAsyst™ or Enzfit (Biosoft, Cambridge, UK) can facilitate the determination of kinetic parameters from catalytic rate data.

For enzymes such as polymerases that have multiple substrates, varying the concentration of only one substrate while holding the others constant typically yields normal Michaelis-Menten kinetics.

For a more thorough discussion of enzyme kinetics, see, e.g., Berg, Tymoczko, and Stryer (2002) Biochemistry, Fifth Edition, W. H. Freeman; Creighton (1984) Proteins: Structures and Molecular Principles, W. H. Freeman; and Fersht (1985) Enzyme Structure and Mechanism, Second Edition, W. H. Freeman.

### Surfaces and Binding Partners

The surfaces of the invention can present a solid or semi-solid surface for any of a variety of linking chemistries that permit coupling of the binding partner to the surface. The binding partners coupled to the surfaces can be any of those noted herein, e.g., any partner that binds a surface coupling domain.

A wide variety of organic and inorganic materials, both natural and synthetic may be employed as the material for the surface. Illustrative organic materials include, e.g., polymers such as polyethylene, polypropylene, poly(4-methylbutene), polystyrene, polymethylmethacrylate (PMMA), polyethylene terephthalate), rayon, nylon, poly(vinyl butyrate), polyvinylidene difluoride (PVDF), silicones, polyformaldehyde, cellulose, cellulose acetate, nitrocellulose, and the like. Other materials that may be employed as the surfaces or components thereof, include papers, ceramics, glass, metals, metalloids, semiconductive materials, cements, or the like. In addition, substances that form gels, such as proteins (e.g., gelatins), lipopolysaccharides, silicates, and agarose are also optionally used.

In several embodiments, the solid surface is a planar, substantially planar, or curved surface such as an array chip, a wall of an enzymatic reaction vessel such as a sequencing or amplification chamber, or the like.

A wide variety of linking chemistries are available for linking molecules constituting the binding partners to a wide variety of solid or semi-solid particle support elements. It is impractical and unnecessary to describe all of the possible known linking chemistries for linking molecules to a solid support. It is expected that one of skill can easily select appropriate chemistries, depending on the intended application.

In one preferred embodiment, the surfaces of the invention comprise silicate elements (e.g., glass or silicate surfaces). A variety of silicon-based molecules appropriate for functionalizing such surfaces are commercially available. See, for example, Silicon Compounds Registry and Review, United Chemical Technologies, Bristol, Pa. Additionally, the art in this area is very well developed and those of skill will be able to choose an appropriate molecule for a given purpose. Appropriate molecules can be purchased commercially, synthesized de novo, or can be formed by modifying an available molecule to produce one having the desired structure and/or characteristics.

The binding partner attaches to the solid substrate through any of a variety of chemical bonds. For example, the linker is optionally attached to the solid substrate using carbon-carbon bonds, for example via substrates having (poly)trifluorochloroethylene surfaces, or siloxane bonds (using, for example, glass or silicon oxide as the solid substrate). Siloxane bonds with the surface of the substrate are formed in one embodiment via reactions of derivatization reagents bearing trichlorosilyl or trialkoxysilyl groups. The particular linking group is selected based upon, e.g., its hydrophilic/hydrophobic properties where presentation of the binding partner in solution is desirable. Groups which are suitable for attachment to a linking group include amine, hydroxyl, thiol, carboxylic acid, ester, amide, isocyanate and isothiocyanate. Preferred derivatizing groups include aminoalkyltrialkoxysilanes, hydroxyalkyltrialkoxysilanes, polyethyleneglycols, polyethyleneimine, polyacrylamide, polyvinylalcohol and combinations thereof.

The binding partners that can be attached to a derivitized surface by these methods include peptides, nucleic acids, mimetics, large and small organic molecules, polymers and the like. The amino acids that are coupled in polypeptide binding partners can be either those having a structure which occurs naturally or they can be of unnatural structure (i.e., synthetic or unnatural, e.g., produced in a system of orthogonal components as noted above). Useful naturally occurring amino acids for coupling include, arginine, lysine, aspartic acid and glutamic acid. Surfaces that bind combinations of these amino acids are also of use in the present invention. Further, peptides comprising one or more residues having a charged or potentially charged side chain are useful binding partner components; these can be synthesized utilizing arginine, lysine, aspartic acid, glutamic acid and combinations thereof. Useful unnatural amino acids are commercially available or can be synthesized utilizing art-recognized methods. In those embodiments in which an amino acid moiety having an acidic or basic side chain is used, these moieties can be attached to a surface bearing a reactive group through standard peptide synthesis methodologies or easily accessible variations thereof. See, for example, Jones (1992), Amino Acid and Peptide Synthesis, Oxford University Press, Oxford.

Linking groups can also be incorporated into the binding partners of the invention. Linking groups of use in the present invention can have any of a range of structures, substituents and substitution patterns. They can, for example, be derivitized with nitrogen, oxygen and/or sulfur containing groups which are pendent from, or integral to, the linker group backbone. Examples include, polyethers, polyacids (polyacrylic acid, polylactic acid), polyols (e.g., glycerol), polyamines (e.g., spermine, spermidine) and molecules having more than one nitrogen, oxygen and/or sulfur moiety (e.g., 1,3-diamino-2-propanol, taurine). See, for example, Sandler et al. (1983) Organic Functional Group Preparations 2nd Ed., Academic Press, Inc. San Diego. A wide range of mono-, di- and bis-functionalized poly(ethyleneglycol) molecules are commercially available and will prove generally useful in this aspect of the invention. See, for example, 1997-1998 Catalog, Shearwater Polymers, Inc., Huntsville, Ala. Additionally, there are a number of easily practiced, useful modification strategies that can be applied to making linkers. See, for example, Harris, (1985) Rev. Macromol. Chem. Phys., C25(3), 325-373; Zalipsky et al., (1983) Eur. Polym. J., 19(12), 1177-1183; U.S. Pat. No. 5,122,614, issued Jun. 16, 1992 to Zalipsky; U.S. Pat. No. 5,650,234, issued to Dolence et al. Jul. 22, 1997, and references therein.

In a preferred embodiment of the invention, the coupling chemistries for coupling binding partners to the surfaces of the invention are light-controllable, i.e., utilize photo-reactive chemistries. The use of photo-reactive chemistries and masking strategies to activate binding partner coupling to surfaces, as well as other photo-reactive chemistries is generally known (e.g., for semi-conductor chip fabrication and for coupling bio-polymers to solid phase materials). The use of photo-cleavable protecting groups and photo-masking permits type switching of both mobile and fixed array members, i.e., by altering the presence of substrates present on the array members (i.e., in response to light). Among a wide variety of protecting groups which are useful are nitroveratryl (NVOC)-methylnitroveratryl (Menvoc), allyloxycarbonyl (ALLOC), fluorenylmethoxycarbonyl (FMOC), -methylnitro-piperonyloxycarbonyl (MeNPOC), —NH-FMOC groups, t-butyl esters, t-butyl ethers, and the like. Various exemplary protecting groups (including both photo-cleavable and non-photo-cleavable groups) are described in, for example, Atherton et al., (1989) Solid Phase Peptide Synthesis, IRL Press, and Greene, et al. (1991) Protective Groups In Organic Chemistry, 2nd Ed., John Wiley & Sons, New York, N.Y., as well as, e.g., Fodor et al. (1991) Science, 251: 767-777, Wang (1976) J. Org. Chem. 41: 3258; and Rich, et al. (1975) J. Am. Chem. Soc. 97: 1575-1579.

Libraries

Enzymes bound to solid surfaces as described above can be formatted into libraries. The precise physical layout of these libraries is at the discretion of the practitioner. One can conveniently utilize gridded arrays of library members (e.g., individual bound enzymes, or blocks of enzyme types bound at fixed locations), e.g., on a glass or polymer surface, or formatted in a microtiter dish or other reaction vessel, or even dried on a substrate such as a membrane. However, other layout arrangements are also appropriate, including those in which the library members are stored in separate locations that are accessed by one or more access control elements (e.g., that comprise a database of library member locations). The library format can be accessible by conventional robotics or microfluidic devices, or a combination thereof.

One common array format for use is a microtiter plate array, in which the library comprises an array embodied in the wells of a microtiter tray (or the components therein). The surfaces of the microtiter tray, or of beads located in the microtiter tray provide two convenient implementations of libraries of surface-bound enzymes. Such trays are commercially available and can be ordered in a variety of well sizes and numbers of wells per tray, as well as with any of a variety of functionalized surfaces for binding of binding partners. Common trays include the ubiquitous 96 well plate, with 384 and 1536 well plates also in common use.

In addition to libraries that comprise liquid phase components, the libraries can also simply comprise solid phase arrays of enzymes (e.g., that can have liquid phase reagents added to them during operation). These arrays fix enzymes in a spatially accessible pattern (e.g., a grid of rows and columns) onto a solid substrate such as a membrane (e.g., nylon or nitrocellulose), a polymer or ceramic surface, a glass or modified silica surface, a metal surface, or the like.

While component libraries are most often thought of as physical elements with a specified spatial-physical relationship, the present invention can also make use of “logical” libraries, which do not have a straightforward spatial organization. For example, a computer system can be used to track the location of one or several components of interest which are located in or on physically disparate components. The computer system creates a logical library by providing a “look-up” table of the physical location of array members (e.g., using a commercially available inventory tracking system). Thus, even components in motion can be part of a logical library, as long as the members of the library can be specified and located.

Single Molecule Detection

The detection of activity of a single molecule of enzyme, or of a few proximal molecules, has a number of applications. For example, single molecule detection in sequencing applications can be used to dramatically reduce reagent consumption and to increase sequencing throughput. Detection of single molecule activity or of low numbers of molecules can similarly be used to reduce reagent consumption in other enzymatic assays.

In one example reaction of interest, a polymerase reaction can be isolated within an extremely small observation volume that effectively results in observation of individual polymerase molecules. As a result, the incorporation event provides observation of an incorporating nucleotide analog that is readily distinguishable from non-incorporated nucleotide analogs. In a preferred aspect, such small observation volumes are provided by immobilizing the polymerase enzyme within an optical confinement, such as a Zero Mode Waveguide (ZMW). For a description of ZMWs and their application in single molecule analyses, and particularly nucleic acid sequencing, see, e.g., Levene et al., Zero-mode waveguides for single-molecule analysis at high concentrations, Science 299:682-686 (2003), Published U.S. Patent Application No. 2003/0044781, and U.S. Pat. No. 6,917,726, each of which is incorporated herein by reference in its entirety for all purposes.

In one aspect, the enzyme (e.g., polymerase) includes a label, e.g., a fluorescent label. Such a label is optionally used to track the position of the enzyme in a ZMW. The label can be attached to the enzyme by any of a number of techniques known in the art; as just one example, an enzyme including a SNAP-tag can be labeled with a fluorophore by reaction with SNAP-vitro 488 or a similar compound (see, e.g., www(dot)covalys(dot)com).

### Kits

Kits of the invention can take any of several different forms. For example, the surface bound enzymes can be provided as components of the kits, or the surface can be provided with binding partners suitable to bind the enzymes, which are optionally packaged separately. The kits can include packaging materials suitable to the application, e.g., with the enzymes of the invention packaged in a fashion to enable use of the enzymes. Regents that are relevant to enzyme function are optionally included as components of the kit, e.g., enzyme substrates, reaction buffers, or the like. Instructions for making or using surface bound enzymes are an optional feature of the invention.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

### Example 1

### Multiple Surface Coupling Domains Provide Higher Binding Affinity

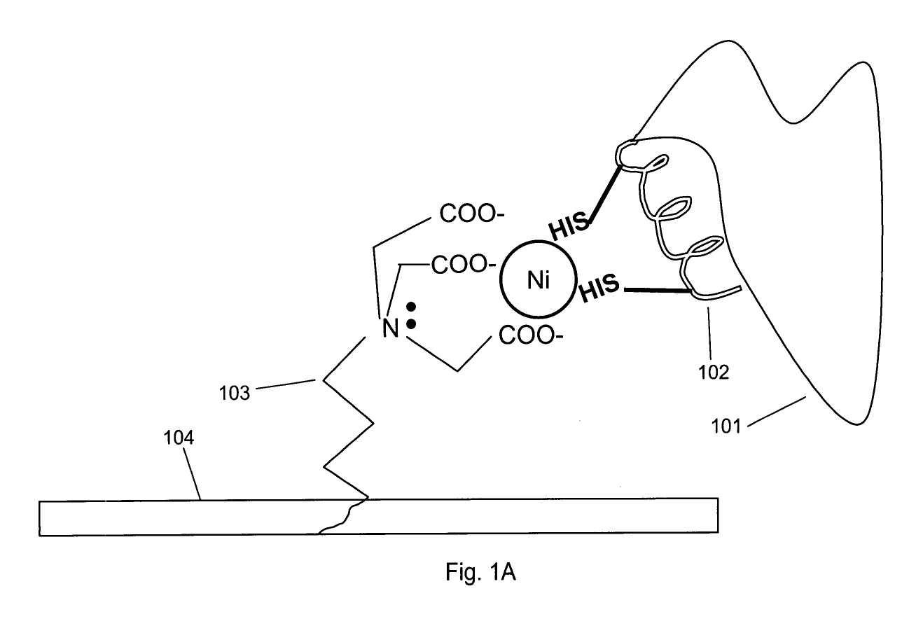

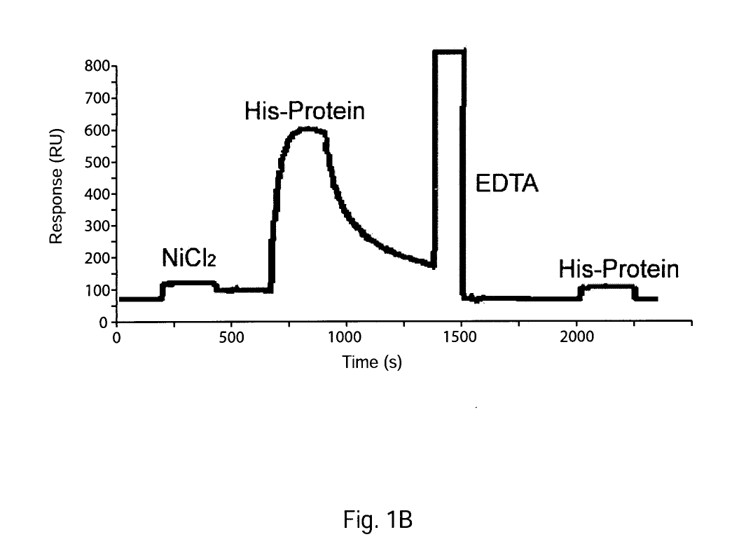

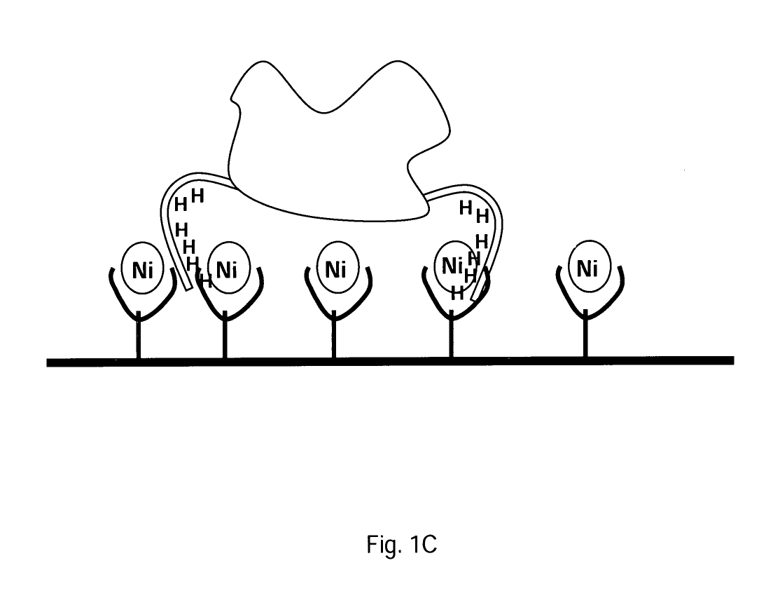

Interaction of a protein bearing a single His-6 tag with nickel-NTA (Ni²⁺-nitrilotriacetic acid) is schematically illustrated in FIG. 1 Panel A. NTA 103 is immobilized on surface 104. Two histidine residues from His-6 tag 102 on protein 101 participate in coordinating the nickel ion.

Surface plasmon resonance detection of the interaction between such a singly His-tagged protein and a sensor chip bearing immobilized nickel-NTA is illustrated by the BIAcore® sensorgram showed in FIG. 1 Panel B (sensorgram from home (dot) hccnet (dot) nl/ja (dot) marquart/Sensorchips/NTA/NTA (dot) htm). From the t_{1/2} of the decay, k_{off} for the dissociation of the singly tagged protein is estimated to be 1×10⁻²s⁻¹.

Nieba et al. (1997) “BIACORE analysis of histidine-tagged proteins using a chelating NTA sensor chip” Analytical Biochemistry 252:217-228 describe BIAcore® analysis of the interaction between various His-tagged protein constructs and a nickel-NTA sensor chip. A protein bearing a single His tag has a K_{d} of 1×10⁻⁶ M⁻¹ and a k_{off} similar to that noted above (i.e., about 1×10⁻² s⁻¹). When multiple His tags are present on a single protein, however, k_{off} becomes dramatically slower (e.g., much less than 1×10⁻⁴ s⁻¹), illustrating that binding of a protein to a surface through two or more surface coupling domains (e.g., multiple His tags, as in FIG. 1 Panel C) results in a higher binding affinity than does binding of the protein to the surface through a single surface coupling domain (e.g., a single His tag).

### Example 2

### Recombinant Enzymes

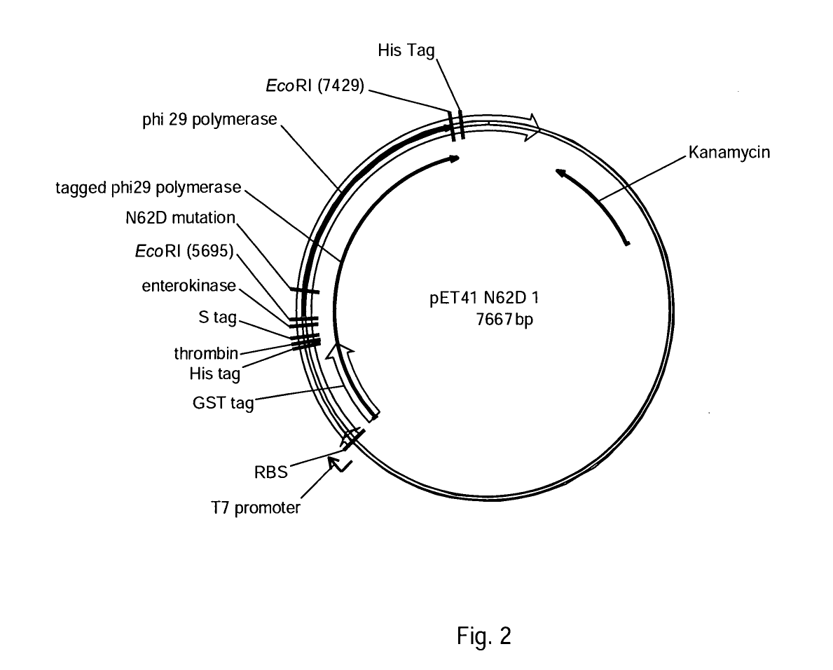

A vector for expression of a recombinant Phi 29 polymerase with three different surface coupling domains was constructed and is schematically illustrated in FIG. 2. An N62D mutation was introduced into wild-type Phi 29 to reduce exonuclease activity. As will be appreciated, the numbering of amino acid residues is with respect to the wild-type sequence of the Phi 29 polymerase, and actual position within a molecule of the invention may vary based upon the nature of the various modifications that the enzyme includes relative to the wild type Phi 29 enzyme, e.g., deletions and/or additions to the molecule, either at the termini or within the molecule itself. GST (glutathione-S-transferase), His, and S tags were added as surface coupling domains. Sequences of the resulting tagged N62D Phi 29 enzyme and of the vector are presented in U.S. patent application 60/753,670 entitled “Polymerases for nucleotide analogue incorporation” by Hanzel et al., filed Dec. 22, 2005, and incorporated herein by reference in its entirety. The tagged N62D Phi 29 polymerase is encoded by nucleotides 4839-7428 of the vector sequence, with the polymerase at nucleotides 5700-7428 and the N62D mutation at nucleotides 5883-5885. The GST, His, and S tag surface coupling domains are encoded by nucleotides 4839-5699. Other features of the vector include the ribosome binding site (nucleotides 4822-4829), T7 promoter (nucleotides 4746-4758), and kanamycin resistance marker (complement of nucleotides 563-1375).

Additional mutations are readily introduced into this construct as desired, for example, to facilitate expression of recombinant Phi 29 polymerases having one or more of: a K135A mutation, an E375H mutation, an E375S mutation, an E375K mutation, an E375R mutation, an E375A mutation, an E375Q mutation, an E375W mutation, an E375Y mutation, an E375F mutation, an L384R mutation, an E486A mutation, an E486D mutation, a K512A mutation, a deletion of the NipTuck domain (residues 505-525), and a deletion within the NipTuck domain. For exemplary amino acid and nucleotide sequences including or encoding such mutations, see Attorney Docket number 105-001310US “POLYMERASES FOR NUCLEOTIDE ANALOGUE INCORPORATION” by Hanzel et al., co-filed herewith, and U.S. patent application 60/753,670 entitled “POLYMERASES FOR NUCLEOTIDE ANALOGUE INCORPORATION” by Hanzel et al., filed Dec. 22, 2005. Similarly, wild-type Phi 29 having GST, His, and S tag surface coupling domains can be expressed from a similar construct.

The recombinant polymerase can be expressed in E. coli, for example, and purified using the GST, His, and/or S tags and standard techniques. The recombinant polymerase is optionally bound to a surface through one or more of the surface coupling domains. One or more of the GST, His, and S tags is optionally removed by digestion with an appropriate protease (e.g., thrombin or enterokinase, whose sites flank the S tag in the construct described above), for example, either following purification of the polymerase prior to coupling of the polymerase to a surface, or after coupling the polymerase to the surface in order to release the polymerase from the surface.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

What is claimed is:

1. An isolated or recombinant enzyme comprising a plurality of artificial or recombinant surface coupling domains, wherein the enzyme, when coupled to a surface through the surface coupling domains, is enzymatically active.

2. The isolated or recombinant enzyme of claim 1, wherein the enzyme is selected from: a polymerase, a DNA polymerase, an RNA polymerase, a reverse transcriptase, a helicase, a kinase, a caspase, a phosphatase, a terminal transferase, an endonuclease, an exonuclease, a dehydrogenase, a peptidase, a beta-lactamase, a beta-galactosidase, and a luciferase.

3. The isolated or recombinant enzyme of claim 2, wherein the enzyme is a polymerase homologous to: a Taq polymerase, an exonuclease deficient Taq polymerase, an E. coli DNA Polymerase 1, a Klenow fragment, a reverse transcriptase, a Φ29 related polymerase, a wild type Φ29 polymerase, an exonuclease deficient Φ29 polymerase, a T7 DNA Polymerase, a T5 DNA Polymerase; or
wherein the enzyme is a polymerase homologous to a Φ29 DNA polymerase, and comprises a structural modification relative to the Φ29 DNA polymerase selected from: a deletion of residues 505-525, a deletion within residues 505-525, a K135A mutation, an E375H mutation, an E375S mutation, an E375K mutation, an E375R mutation, an E375A mutation, an E375Q mutation, an E375W mutation, an E375Y mutation, an E375F mutation, an L384R mutation, an E486A mutation, an E486D mutation, a K512A mutation, an N62D mutation, a D12A mutation, and combinations thereof.

4. The isolated or recombinant enzyme of claim 1, wherein at least one of the artificial surface coupling domains comprise: a recombinant dimer domain of the enzyme, a large extraneous polypeptide domain, a polyhistidine tag, a HIS-6 tag, a biotin, an avidin sequence, a GST sequence, a glutathione, a AviTag sequence, an S tag, an antibody, an antibody domain, an antibody fragment, an antigen, a receptor, a receptor domain, a receptor fragment, a ligand, a dye, an acceptor, a quencher, or a combination thereof.

5. The isolated or recombinant enzyme of claim 1, comprising at least two different artificial coupling domains that are specifically bound by at least two different cognate binding components.

6. The isolated or recombinant enzyme of claim 1, comprising at least three different artificial coupling domains that are specifically bound by at least three different cognate binding components.

7. The isolated or recombinant enzyme of claim 1, wherein the enzyme comprises 3 or more artificial or recombinant surface coupling domains.

8. The isolated or recombinant enzyme of claim 1, wherein the enzyme comprises 5 or more artificial or recombinant surface coupling domains.

9. The isolated or recombinant enzyme of claim 1, wherein the enzyme comprises 10 or more artificial or recombinant surface coupling domains.

10. The isolated or recombinant enzyme of claim 1, wherein at least one of the artificial surface coupling domains comprises a purification tag.

11. The isolated or recombinant enzyme of claim 1, wherein the artificial surface coupling domains are distal to an active site of the enzyme.

12. The isolated or recombinant enzyme of claim 1, wherein the active site is located within a C-terminal domain of the enzyme, and the artificial surface coupling domain is located within an N-terminal domain of the enzyme.

13. The isolated or recombinant enzyme of claim 1, wherein binding of the enzyme to a surface through the surface coupling domains orients the enzyme relative to the surface.

14. The isolated or recombinant enzyme of claim 1, wherein binding of the enzyme to a surface through at least two of the surface coupling domains has a higher binding affinity than binding of the enzyme to the surface through a single surface coupling domain.

15. The isolated or recombinant enzyme of claim 1, wherein the enzyme, when bound to the surface, retains a k_{cat}/Kₘ that is at least 1% as high as the enzyme in solution.

16. The isolated or recombinant enzyme of claim 1, wherein the enzyme, when bound to the surface, retains a k_{cat}/Kₘ that is at least 10% as high as the enzyme in solution.

17. The isolated or recombinant enzyme of claim 1, wherein the enzyme, when bound to the surface, retains a k_{cat}/Kₘ that is at least 50% as high as the enzyme in solution.

18. The isolated or recombinant enzyme of claim 1, wherein the enzyme, when bound to the surface, retains a k_{cat}/Kₘ that is at least 75% as high as the enzyme in solution.

19. A surface comprising an active enzyme bound thereon, wherein the enzyme is coupled to the surface through a plurality of artificial or recombinant surface coupling domains, and wherein the active enzyme displays a k_{cat}/Kₘ that is at least 10% as high as a corresponding active enzyme in solution.

20. The surface of claim 19, wherein a location of the enzyme on the surface is fixed, thereby providing a spatial address of the enzyme on the surface.

21. The surface of claim 19, wherein the surface is a planar surface.

22. The surface of claim 19, wherein the surface comprises a polymer, a ceramic, glass, a bead, a microbead, a polymer bead, a glass bead, a well, a microwell, a slide, a grid, a rotor, a microchannel, or a combination thereof.

23. The surface of claim 54, wherein the surface comprises or is proximal to a Zero Mode Wave Guide.

24. The surface of claim 19, wherein the surface comprises one or more immobilized components selected from: a dye, an acceptor, a quencher, an immobilized metal, an immobilized glutathione, an immobilized antibody, an immobilized antibody fragment, an immobilized antigen, an immobilized receptor, an immobilized receptor fragment, an immobilized ligand, an immobilized hapten, an immobilized biotin, an immobilized avidin, an immobilized GST sequence, glutathione, an immobilized AviTag sequence, an immobilized S tag, an immobilized S protein, and a combination thereof; and
wherein the surface coupling domains specifically bind to the immobilized components.

25. The surface of claim 19, wherein the surface coupling domains comprise at least two different domains and wherein the immobilized component comprises at least two different immobilized components.

26. The surface of claim 25, wherein the at least two different domains are concurrently bound to the at least two different immobilized components.

27. The surface of claim 19, wherein the surface coupling domains are distal to an active site of the enzyme.

28. The surface of claim 19, wherein the k_{cat}/Kₘ that is at least 50% as high as a corresponding active enzyme in solution.

29. The surface of claim 19, wherein the K_{cat}/Kₘ that is at least 75% as high as a corresponding active enzyme in solution.

30. The surface of claim 19, wherein the active site is located within a C-terminal domain of the enzyme, and the artificial surface coupling domain is located within an N-terminal domain of the enzyme.

31. The surface of claim 19, wherein binding of the enzyme to the surface through the surface coupling domains orients the enzyme relative to the surface.

32. The surface of claim 19, wherein binding of the enzyme to the surface through at least two of the surface coupling domains has a higher binding affinity than binding of the enzyme to the surface through a single surface coupling domain.

33. The surface of claim 19, wherein the enzyme is selected from: a polymerase, a DNA polymerase, an RNA polymerase, a reverse transcriptase, a helicase, a kinase, a caspase, a phosphatase, a terminal transferase, an endonuclease, an exonuclease, a dehydrogenase, a peptidase, a beta-lactamase, a beta-galactosidase, and a luciferase; or
wherein the enzyme is a polymerase homologous to: a Taq polymerase, an exonuclease deficient Taq polymerase, an E. coli DNA Polymerase 1, a Klenow fragment, a reverse transcriptase, a Φ29 related polymerase, a wild type Φ29 polymerase, an exonuclease deficient Φ29 polymerase, a T7 DNA Polymerase, a T5 DNA Polymerase; or
wherein the enzyme is a polymerase homologous to a Φ29 DNA polymerase, and comprises a structural modification relative to the Φ29 DNA polymerase selected from: a deletion of residues 505-525, a deletion within residues 505-525, a K135A mutation, an E375H mutation, an E375S mutation, an E375K mutation, an E375R mutation, an E375A mutation, an E375Q mutation, an E375W mutation, an E375Y mutation, an E375F mutation, an L384R mutation, an E486A mutation, an E486D mutation, a K512A mutation, an N62D mutation, a D12A mutation, and combinations thereof.

34. A method of binding an enzyme to a surface, the method comprising:
providing an isolated or recombinant enzyme comprising a plurality of artificial or recombinant surface coupling domains;
providing a surface comprising a plurality of binding partners that specifically bind to the surface coupling domains;
contacting the enzyme to the surface; and,
permitting the binding partners to bind to the surface coupling domains, thereby binding the enzyme to the surface.

35. The method of claim 34, further comprising releasing the enzyme from the surface subsequent to binding the enzyme to the surface.

36. The method of claim 34, wherein the surface coupling domain is activateable.

37. The method of claim 34, wherein the surface coupling domain is caged and the method includes uncaging the surface coupling domain, thereby permitting the binding partners to bind to the surface coupling domains.

38. The method of claim 34, wherein the surface coupling domain is photocaged and the method includes uncaging the surface coupling domain, thereby permitting the binding partners to bind to the surface coupling domains.

39. An isolated or recombinant active site-containing protein comprising: an artificial or recombinant surface coupling domain that is at least 5 kDa in size, wherein the protein, when coupled to a surface through the surface coupling domain, retains at least 1% activity as compared to an activity of a corresponding active protein in solution.

40. The isolated of recombinant protein of claim 39, wherein the protein retains at least 10% activity.

41. The isolated of recombinant protein of claim 39, wherein the surface coupling domain is at least 10 kDa.

42. The isolated or recombinant protein of claim 39, wherein the surface coupling domain is at least 20 kDa.

43. The isolated or recombinant protein of claim 39, wherein the surface coupling domain is at least 50 kDa.

44. The isolated or recombinant protein of claim 39, wherein the surface coupling domain is at least 100 kDa.

45. The isolated or recombinant protein of claim 39, wherein the surface coupling domain is at least 1000 kDa.

46. The isolated or recombinant protein of claim 39, wherein the surface coupling domain comprises one or more of: a recombinant dimer domain of the protein, a large extraneous polypeptide domain, a polyhistidine tag, a HIS-6 tag, a biotin, an avidin sequence, a GST sequence, a glutathione, a AviTag sequence, an S tag, an antibody, an antibody domain, an antibody fragment, an antigen, a receptor, a receptor fragment, a ligand, a dye, an acceptor, a quencher, or a combination thereof.

47. The isolated or recombinant protein of claim 39, wherein the protein comprises at least two surface coupling domains.

48. The isolated or recombinant protein of claim 39, wherein the protein comprises three or more surface coupling domains.

49. The isolated or recombinant protein of claim 39, wherein binding of the protein to the surface through the surface coupling domain specifically orients the protein relative to the surface.

50. The isolated or recombinant protein of claim 39, wherein the protein is selected from: an enzyme, a receptor, an antibody, a polymerase, a DNA polymerase, an RNA polymerase, a reverse transcriptase, a helicase, a kinase, a caspase, a phosphatase, a terminal transferase, an endonuclease, an exonuclease, a dehydrogenase, a peptidase, a beta-lactamase, a beta-galactosidase, and a luciferase.

51. The isolated or recombinant protein of claim 39, wherein the protein is a polymerase homologous to: a Phi29 DNA polymerase, a Taq polymerase, an exonuclease deficient Taq polymerase, an E. coli DNA Polymerase 1, a Klenow fragment, a reverse transcriptase, a Φ29 related polymerase, a wild type Φ29 polymerase, an exonuclease deficient Φ29 polymerase, a T7 DNA Polymerase, a T5 DNA Polymerase; or
wherein the enzyme is a polymerase homologous to a Φ29 DNA polymerase, and comprises a structural modification relative to the Φ29 DNA polymerase selected from: a deletion of residues 505-525, a deletion within residues 505-525, a K135A mutation, an E375H mutation, an E375S mutation, an E375K mutation, an E375R mutation, an E375A mutation, an E375Q mutation, an E375W mutation, an E375Y mutation, an E375F mutation, an L384R mutation, an E486A mutation, an E486D mutation, a K512A mutation, an N62D mutation, a D12A mutation, and combinations thereof.

52. The isolated or recombinant protein of claim 39, wherein the protein retains at least 50% activity when coupled to the surface.

53. The isolated or recombinant protein of claim 39, wherein the protein retains at least 75% activity when coupled to the surface.

54. A surface comprising a protein bound thereon, wherein the protein is coupled to the surface through an artificial or recombinant surface coupling domain that is at least 5 kDa in size, wherein the protein displays an activity that is at least 10% as high as a corresponding active protein in solution.

55. The surface of claim 54, wherein a location of the enzyme on the surface is fixed, thereby providing a spatial address of the enzyme on the surface.

56. The surface of claim 54, wherein the surface is a planar surface.

57. The surface of claim 54, wherein the surface comprises a polymer, a ceramic, glass, a bead, a microbead, a polymer bead, a glass bead, a well, a microwell, a slide, a grid, a rotor, a microchannel, or a combination thereof.

58. The surface of claim 54, wherein the surface comprises or is proximal to a Zero Mode Wave Guide.

59. The surface of claim 54, wherein the surface comprises one or more immobilized component selected from: a dye, an acceptor, a quencher, an immobilized metal, an immobilized glutathione, an immobilized antibody, an immobilized antibody fragment, an immobilized antigen, a an immobilized receptor, a an immobilized receptor fragment, an immobilized ligand, an immobilized hapten, an immobilized biotin, an immobilized avidin, an immobilized GST sequence, a glutathione, an immobilized AviTag sequence, an immobilized S tag, and a combination thereof; and
wherein the surface coupling domain specifically binds to the immobilized component.

60. The surface of claim 59, wherein the protein comprises at least two different surface coupling domains and wherein the surface comprises at least two different immobilized components, each of which specifically bind to at least one of the two different surface coupling domains.

61. The surface of claim 60, wherein the at least two different domains are concurrently bound to the at least two different immobilized components.

62. The surface of claim 54, wherein the surface coupling domain is distal to the active site.

63. The surface of claim 54, wherein activity is at least 50% as high as a corresponding active protein in solution.

64. The surface of claim 54, wherein the activity is at least 75% as high as a corresponding active protein in solution.

65. The surface of claim 54, wherein the active site is located within a C-terminal domain of the protein, and the artificial surface coupling domain is located within an N-terminal domain of the protein.

66. The surface of claim 54, wherein binding of the protein to the surface through the surface coupling domain orients the protein relative to the surface.

67. The surface of claim 54, wherein the protein is selected from: an enzyme, an antibody, a receptor, a polymerase, a DNA polymerase, an RNA polymerase, a reverse transcriptase, a helicase, a kinase, a caspase, a phosphatase, a terminal transferase, an endonuclease, an exonuclease, a dehydrogenase, a peptidase, a beta-lactamase, a beta-galactosidase, and a luciferase; or
wherein the protein is a polymerase homologous to: a Taq polymerase, an exonuclease deficient Taq polymerase, an E. coli DNA Polymerase 1, a Klenow fragment, a reverse transcriptase, a Φ29 related polymerase, a wild type Φ29 polymerase, an exonuclease deficient Φ29 polymerase, a T7 DNA Polymerase, a T5 DNA Polymerase; or
wherein the protein is a polymerase homologous to a Φ29 DNA polymerase, and comprises a structural modification relative to the Φ29 DNA polymerase selected from: a deletion of residues 505-525, a deletion within residues 505-525, a K135A mutation, an E375H mutation, an E375S mutation, an E375K mutation, an E375R mutation, an E375A mutation, an E375Q mutation, an E375W mutation, an E375Y mutation, an E375F mutation, an L384R mutation, an E486A mutation, an E486D mutation, a K512A mutation, an N62D mutation, a D12A mutation, and combinations thereof.

68. A method of binding a protein to a surface, the method comprising:
providing an isolated or recombinant protein comprising an artificial or recombinant surface coupling domain that is at least 5 kDa in size;
providing a surface comprising a binding partner that specifically binds to the surface coupling domain;
contacting the protein to the surface; and,
permitting the binding partner to bind to the surface coupling domain, thereby binding the enzyme to the surface;
wherein the protein, when bound to the surface is at least 10% as active as the protein in solution.

69. The method of claim 68, comprising releasing the protein from the surface subsequent to binding the protein to the surface.

70. The method of claim 68, wherein the surface coupling domain is activateable.

71. The method of claim 68, wherein the surface coupling domain is caged and the method includes uncaging the surface coupling domain, thereby permitting the binding partners to bind to the surface coupling domains.

72. The method of claim 68, wherein the surface coupling domain is photocaged and the method includes uncaging the surface coupling domain, thereby permitting the binding partners to bind to the surface coupling domains.
